(19) United States
(12) Patent Application Publication
(10) Pub. No.: US 2022/0361751 A1
(43) Pub. Date: Nov. 17, 2022

(54) WEARABLE DEVICE AND METHOD FOR REMOTE OPTICAL MONITORING OF INTRAOCULAR PRESSURE

(71) Applicant: Smartlens, Inc., Mountain View, CA (US)

(72) Inventors: Aykutlu DANA, San Jose, CA (US); Savas KOMBAN, Palo Alto, CA (US); Murat BADAY, Palo Alto, CA (US); Sevda AGAOGLU, Santa Clara, CA (US); Ahmet Taylan YAZICI, Palo Alto, CA (US)

(73) Assignee: Smartlens, Inc., Mountain View, CA (US)

(21) Appl. No.: 17/875,719

(22) Filed: Jul. 28, 2022

Related U.S. Application Data
(63) Continuation of application No. PCT/US21/15093, filed on Jan. 26, 2021.
(60) Provisional application No. 62/966,798, filed on Jan. 28, 2020.

(51) Int. Cl. A61B 3/16; A61B 3/00; G02C 7/04

(52) U.S. Cl. CPC A61B 3/16; A61B 3/0025; A61B 3/0008; G02C 7/04; A61B 2562/0223; G02C 2200/02

(57) ABSTRACT

Systems and methods are described for determining an intraocular pressure (IOP) of an eye using a contact lens with a magnet placed on the cornea of the eye. A magnetic field is exerted on the magnet of the contact lens, and the magnet is displaced by the magnetic field. The system of the present disclosure determines the deflection of the cornea based on the magnetic displacement of the magnet and determines the IOP of the eye.

### CROSS-REFERENCE TO RELATED APPLICATIONS

This application is a continuation of PCT Application No. PCT/US2021/15093 (Attorney Docket No. 48675-709601), filed Jan. 26, 2021, which claims benefit of U.S. Provisional Patent Application Ser. No. 62/966,798 (Attorney Docket No. 48675-709.101), filed Jan. 28, 2020, the contents of which is herein incorporated by reference in its entirety.

### FIELD OF THE INVENTION

The present disclosure relates to the technical field of remote optomechanical sensing. More particularly, the technical field of wearable sensor system for measuring the intraocular pressure.

### BACKGROUND OF THE INVENTION

The present disclosure is in the technical fields of optomechanics. More particularly, the present disclosure is in the technical field of intraocular pressure sensing using remote optical measurement of the corneal indentation caused by a magnetic force exerted on a micromagnet embedded in or on a contact lens.

Glaucoma is the second most common cause of blindness in the global world. It is a multifactorial disease with several risk factors, of which intraocular pressure (IOP) is the most important. IOP measurements are used for glaucoma diagnosis and patient monitoring. IOP has wide diurnal fluctuation, and is dependent on body posture, so the occasional measurements done by the eye care expert in clinic can be misleading.

### SUMMARY OF THE INVENTION

The present disclosure describes a wearable optical device and readout methods for measuring the intraocular pressure of an eye.

In an embodiment, there may be a contact lens device to measure an intraocular pressure of an eye. The device has a body formed of an elastomeric material and at least one magnet embedded in or placed on the body. In some embodiments, the elastomeric material may be biocompatible. In some embodiments the material may be transparent.

In an embodiment, there may be an apparatus for reading a contact lens with an embedded magnet. The apparatus has an excitation coil with a driver in electrical communication with the excitation coil. The driver may control the parameters of the coil. A light source may illuminate the contact lens, and an optical sensor may detect the reflected light of the light source. A controller may synchronize the excitation of the coil and the sensor image or data collection. The controller may further have a receiver to receive external data.

In another embodiment, there is a method for determining an IOP reading using an apparatus for reading a contact lens with an embedded magnet. The method provides for exciting an excitation coil with an electric pulse, recording images over a pre-established time period, determining a cornea deformation based on one or more cornea topography images, and calculating the IOP reading using the cornea deformation.

In another embodiment, there may be a system for determining an IOP reading, the system has a base, and an arm extending from the base. A motor may be attached to the arm to vibrate or move the arm. A magnet may be engaged to a distal end of the arm, the magnet may produce a controlled magnetic field. A hall sensor may be attached to a distal end of the magnet. The magnet may generate a directed magnetic field, and the Hall sensor may read the magnetic field, and any perturbations caused by the magnet in the contact lens.

The device may comprise a contact lens with an embedded magnet. A magnetic field may be used to apply force onto the cornea. The changes in the topography of the cornea depends on the applied magnetic force, as well as on corneal parameters and IOP. An external measurement setup, which may be placed on a goggle, may contain any one or more of; a patterned illuminator and optical detector, such as a camera or quadrature photodiode. A change of the corneal topography caused by the application of a time varying magnetic field to the contact lens may be detected using the quadrature photodetector or by analyzing images of a matrix of point illuminators. The mechanical response of the cornea may be measured with different magnetic excitation amplitudes, pulse shapes or waveform frequencies, and frequency response of the cornea may be measured remotely. Indentation depth and resonance frequency changes of the cornea may be calculated using collected data and the IOP may be determined. The method comprises a preliminary characterization of the corneal thickness and topography where the Radius of curvature and corneal thickness may be measured at a known IOP value acquired by conventional ophthalmologic methods. The personalized data set may then used as an input into the data processing algorithms, that also use continuous imaging measurements from the goggle to calculate the IOP. The data may be connected to the cloud and the goggle may also be equipped with a mist generator that may dispense a controlled amount of drugs that may help to reduce the IOP. The present disclosure describes a contact lens and wearable optical device that measures the IOP through optical data acquisition using magnetic excitation of the contact lens. The magnetic excitation of the contact lens may cause deformation of the underlying cornea. One or more light sources, along with a camera, may measure the response to magnetic excitation and use this data along with a reference data for a particular individual to accurately determine the IOP.

### BRIEF DESCRIPTION OF THE DRAWINGS

FIG. 1 illustrates a cross-sectional of a contact lens with an embedded magnet according to an embodiment.

FIG. 2 illustrates a side view of the measurement system and a contact lens, according to an embodiment.

FIG. 3 illustrates a side view of the application of a magnetic field on a contact lens with a magnet according to an embodiment.

FIG. 4 illustrates a graph showing the change in cross-sectional corneal topography with and without the applied magnetic field according to an embodiment.

FIG. 5 illustrates a side view of the measurement system and a contact lens with an embedded magnet, according to an embodiment.

FIG. 6 illustrates a side view of the measurement system and contact lens with an embedded magnet, according to an embodiment.

FIG. 7 illustrates an excitation and optical measurement setup embedded into a goggle according to an embodiment.

FIG. 8 illustrates a magnetic excitation and optical measurement setup embedded into a goggle according to an embodiment.

FIG. 9 illustrates the application of a magnetic field waveform applied to a contact lens according to an embodiment.

FIG. 10 illustrates the application of a periodic pulse-train magnetic field waveform applied to a contact lens according to an embodiment.

FIG. 11 illustrates the application of a periodic pulse-train magnetic field waveform applied to a contact lens, and changes in the output of a position-sensitive photodiode, according to an embodiment.

FIG. 12 illustrates the application of a single pulse magnetic field waveform to a contact lens according to an embodiment.

FIG. 13 illustrates a side view of a measurement system and a contact lens according to an embodiment.

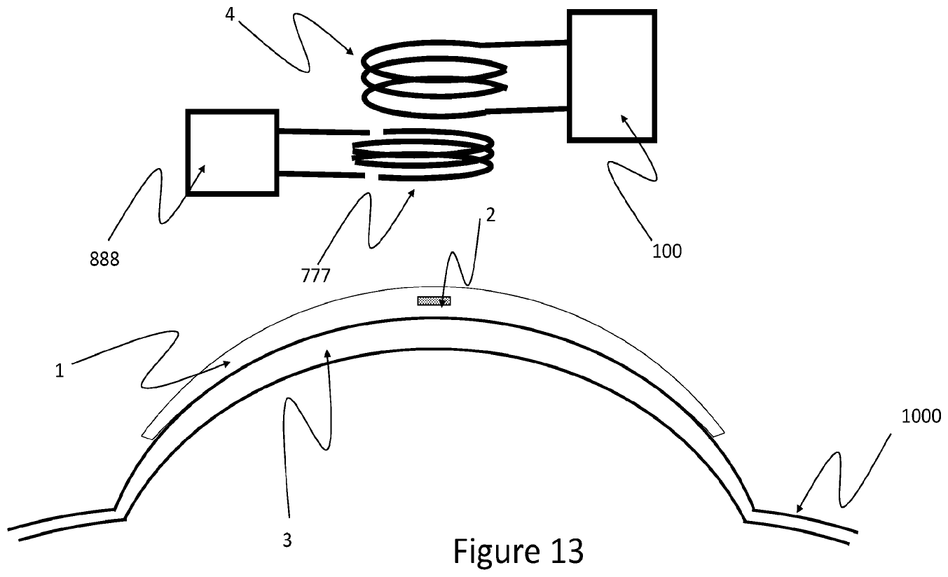

FIG. 14 illustrates an inductive measurement system integrated into a goggle according to an embodiment.

FIG. 15 illustrates a side view of a measurement system according to an embodiment.

### DETAILED DESCRIPTION OF THE INVENTION

Described herein is an optical wearable imaging sensor that monitors the intraocular pressure (IOP) while the user may see through the device and go on with their daily routine. In an embodiment, the device uses personal pre-measured properties of the cornea and users' anatomical parameters, along with measurement results supplied by a measurement device. The operation of the system generally assumes that the corneal topography responds to externally applied forces in a manner that depends on IOP as well as other corneal parameters such as corneal thickness.

The operation of the device may rely on magnetic actuation of the cornea through a contact lens that may have a small magnet embedded into the contact lens. The device may measure, either optically or electronically, the displacement as a function of one or more applied force(s) in amplitude or frequency.

The device may include a substance applicator that may apply a drug to control the IOP based on the measurements done by the device.

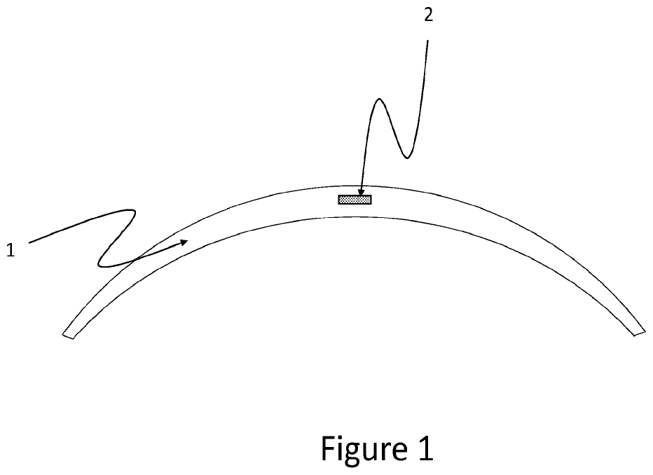

Referring now to the FIG. 1, a contact lens 1 is illustrated with an embedded millimeter or micrometer sized magnet 2. The magnet 2 may be shaped like a disc, a bar, or have a complex shape. The shape may be a regular polygon or an irregular polygon. The magnet may have an aperture or slot through it so the magnet may present a gap space. The magnet may be positioned in or on the contact lens in a manner so as to not interfere with the normal vision of a person. In some embodiments the magnet may be shaped like a ring, so a person's sight may be through the aperture. In some embodiments, one or more magnets may be used to provide different shaped magnetic patterns in the contact lens. In some embodiments a second or several additional magnets may be positioned at different places in or on the contact lens to produce more than one displacement effect in the contact lens when a magnetic field may be exerted on the magnets of the contact lens. In some embodiments there may be more than one magnetic field exerted on one or more of the magnets of the contact lens.

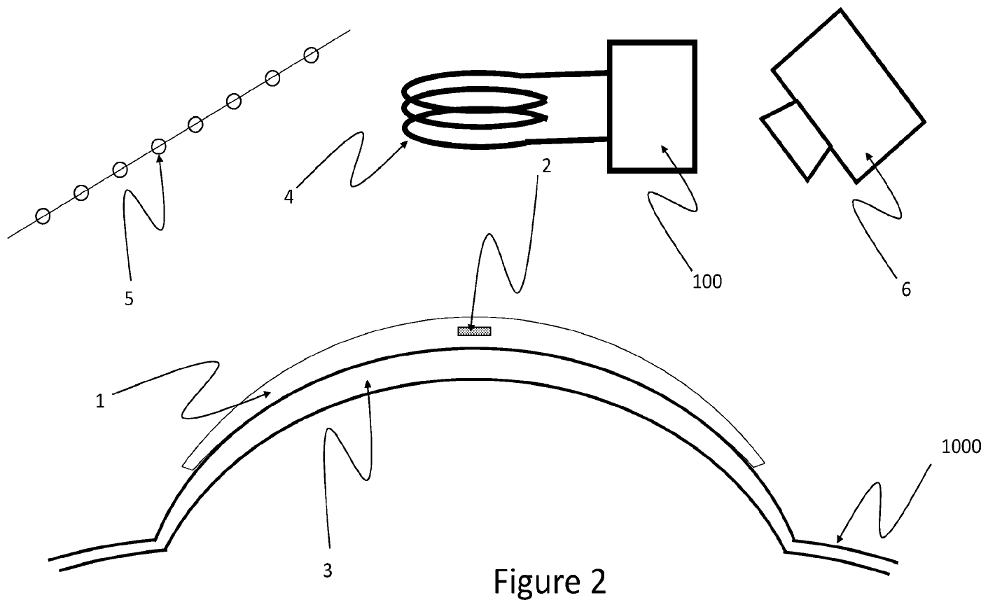

In another embodiment, the contact lens 1 may be placed onto the cornea 3, as shown in FIG. 2. A magnetic excitation coil 4 may be placed in close proximity to the contact lens. The magnetic excitation coil 4 may be controlled by a driver 100 using a computer controlled waveform generator and power amplifier. One or more light sources 5, and a camera 6 may be used to illuminate and capture images (respectively) of the contact lens 2 on the eye 1000.

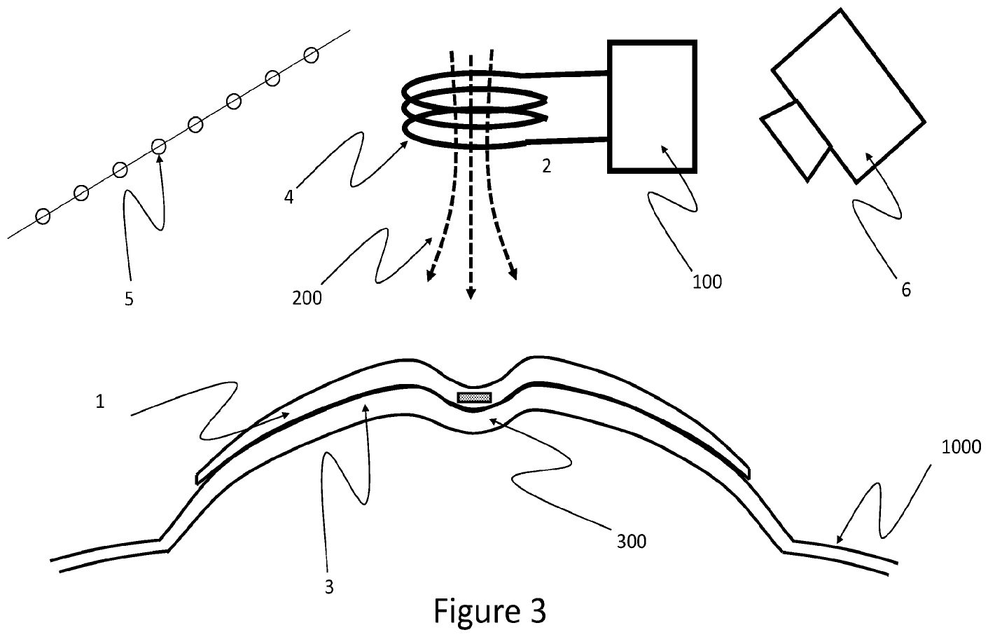

In another embodiment, the contact lens 1 may be placed onto the cornea 3, as shown in FIG. 3. A magnetic excitation coil 4 may be placed in proximity to the contact lens, controlled by a driver 100. A computer controlled waveform generator and power amplifier may be used to control the magnetic excitation coil 4. One or more spot light sources 5, and one or more cameras 6 may be used to illuminate the contact lens on the eye, and take images of it. The coil 4 may be activated to produce a magnetic field 200 which may induce a force on the magnet on or inside the contact lens 1, causing an indentation 300 of the cornea 3. The camera 6 may register the changes in corneal topography and measures the indentation 300. The generation of the magnetic field 200 and the orientation of the magnet 2 may be determined in advance such that the field 200 may cause the repulsion of the magnet 2. In some embodiments the magnetic field 200 may attract the magnet 2, which may provide for a measurement of other aspects of the contact lens, the cornea or the eye.

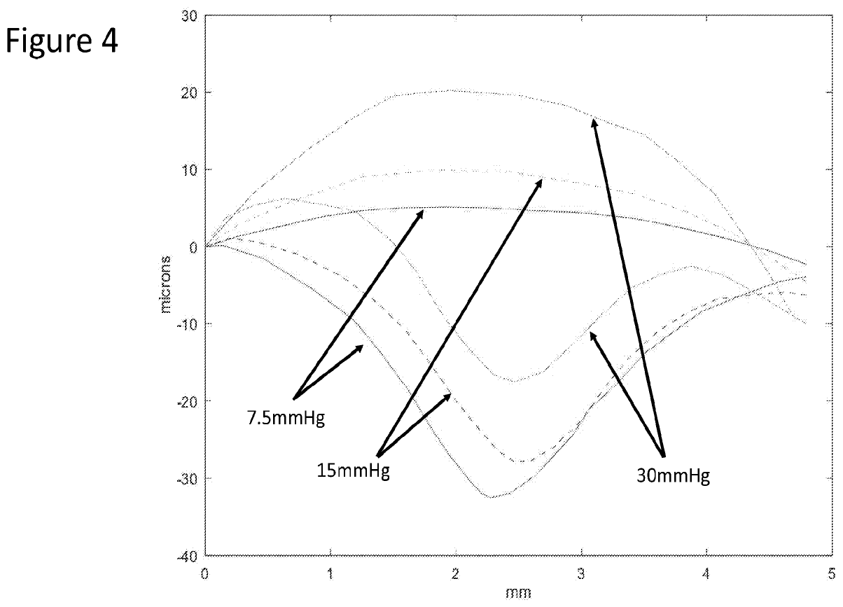

In an embodiment, a graph illustrating a cross sectional of the cornea topography may be seen for IOP values of 7.5 mmHg, 15 mmHg and 30 mmHg (FIG. 4). The graph illustrates the indentation of the cornea with and without a magnetic force where the radius of indentation and apex position of the cornea are modified differently by the magnetic force due to differences in the IOP value. In some embodiments, the negative micron values (Y-Axis), represent the depression of the cornea when the magnet of the contact lens may be repelled by a magnetic field. For low IOP values, a similar magnetic force may induce a wider and smoother indentation. Alternatively, for high IOP values, the indentation may become less pronounced and may occur within a smaller radius. This difference may be used to determine the IOP value through optical or electronic measurement of changes in the cornea topography. The higher IOP values may cause the cornea to have a greater built-in strain and higher resistance to indentation by the magnetic force. In some embodiments, a higher IOP value may result in a smaller but sharper indentation, as compared to a lower IOP value. The exact values of the width of the indentation region as well as the depth of indentation may depend on the cornea thickness as well. The relation between indentation shape and depth can be established through simulations, which may be based on measurements of corneal thickness.

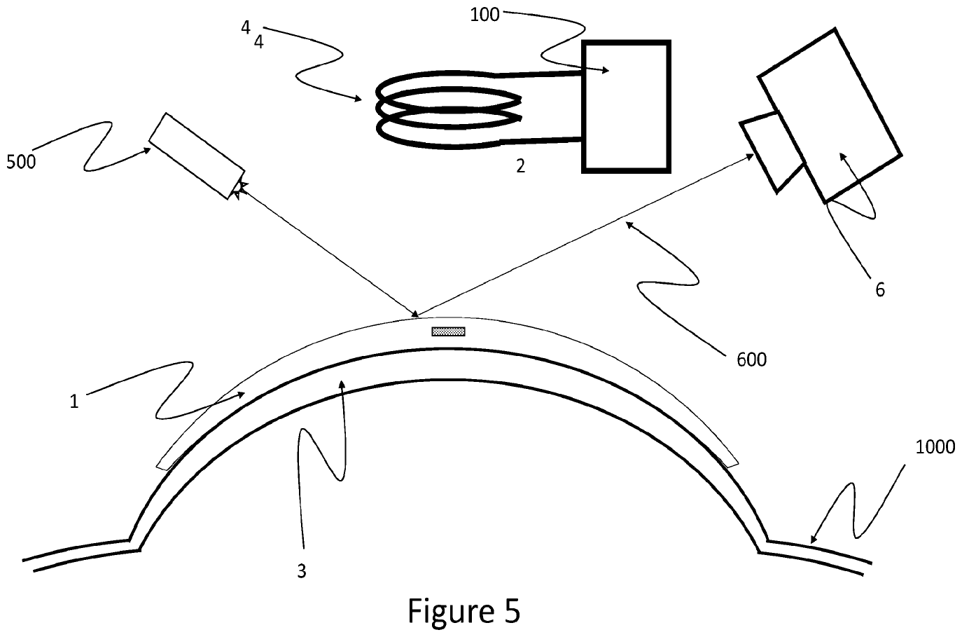

In an embodiment, the contact lens 1 may be placed onto the cornea 3. A magnetic excitation coil 4 may be placed in proximity to the contact lens as shown in FIG. 5. The coil may be controlled by a driver 100 having a computer controlled waveform generator and a power amplifier. A laser may serve as a light source 500 for illuminating the contact lens, and a camera 6 may be used for image capture. The cornea 3 may be indented by the magnetic field from the coil, and changes in the light reflection pattern before and after application of the magnetic field may be recorded by the camera 6. An analysis of the changes in the reflection pattern may be used to extract indentation width and depth information, which may be used to determine the IOP value.

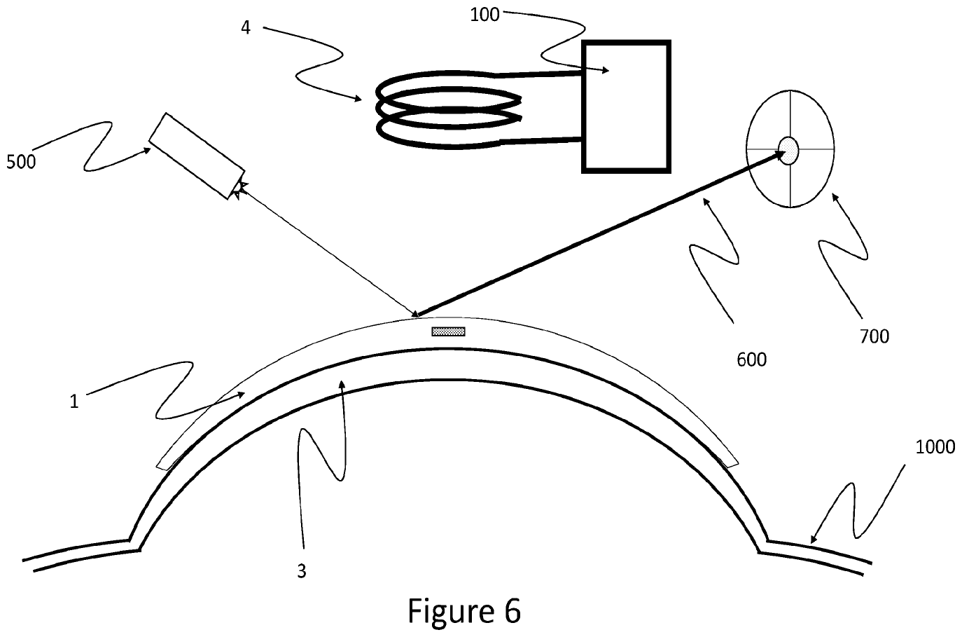

In another embodiment, the contact lens 1 may be placed onto the cornea 3, while a magnetic excitation coil 4 may be placed in proximity to the contact lens, as shown in FIG. 6. The coil may be controlled by a driver 100 using a computer controlled waveform generator and power amplifier, a collimated laser light source 500, and a position sensitive photodetector 700, which may measure the deflection of the reflected laser beam 600. The cornea 3 may be indented by the force exerted on magnet of the lens 1, by the magnetic field from the coil 4, and changes in the reflected beam position are registered through the position sensitive detector 700. An analysis of the changes in the reflection of the beam may be used to extract indentation strength, which may relate to the IOP value.

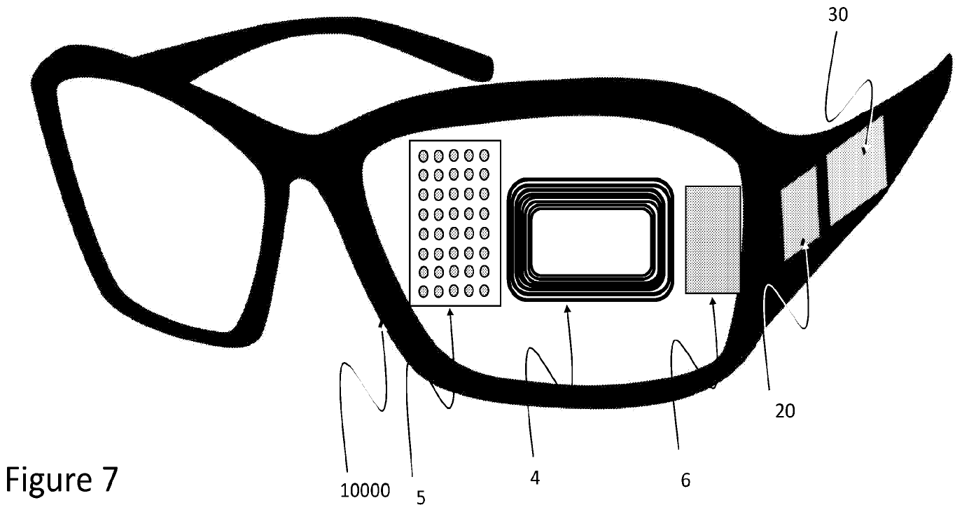

An integration of the measurement setup into a goggle is shown in FIG. 7. In an embodiment, the light sources 5, the camera 6, and the excitation coil 4 may be placed on the goggle 10000, along with an electronic controller 20 and power source/battery pack 30. In some embodiments, the goggles may also have a drug delivery device.

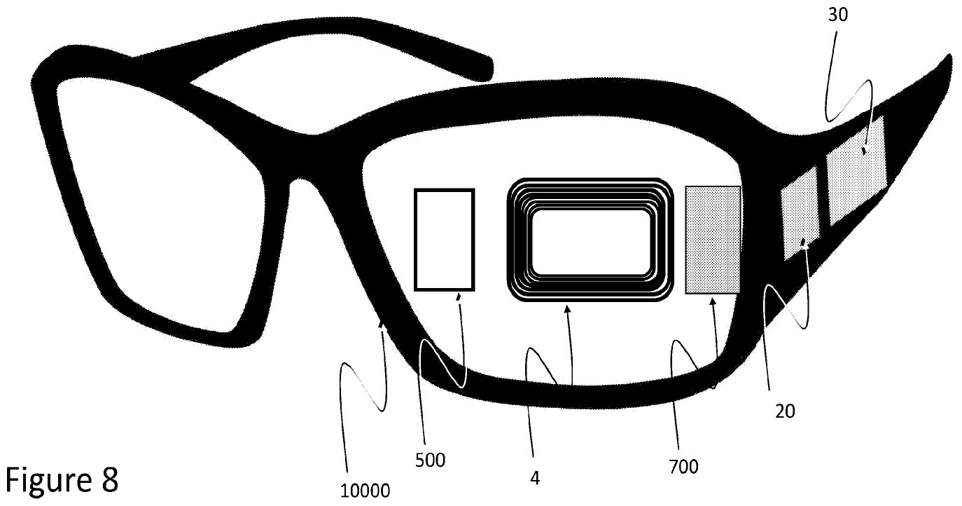

In another embodiment, the goggle may incorporate the laser sources 500, the position sensitive optical detector 700, the excitation coil 4 and an electronic controller 20 and power source/battery pack 30, as shown in FIG. 8.

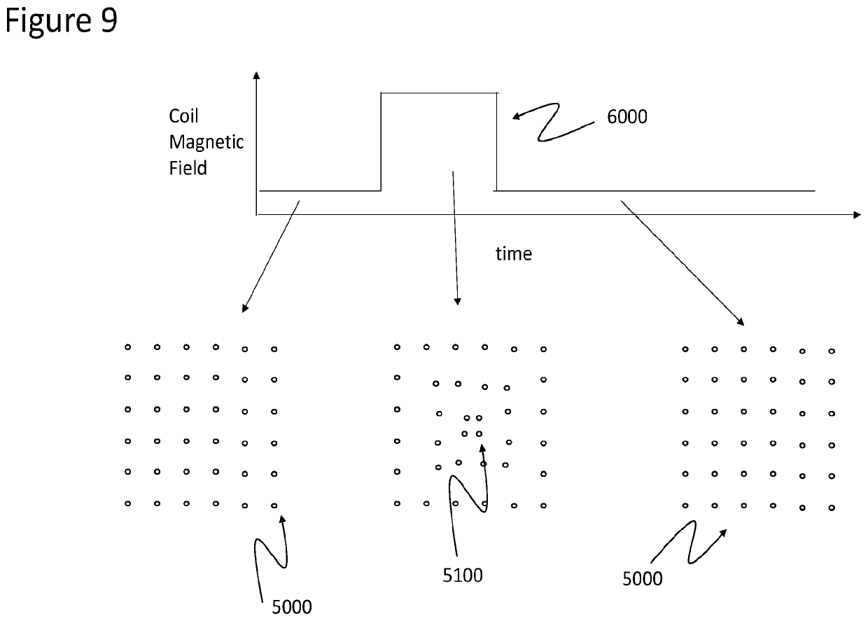

Now referring to FIG. 9, an example measurement of the magnetic force indentation is shown. A magnetic field pulse 6000 may be applied to the contact lens through the excitation coil, while a camera may record one or more images 5000 of reflected light from a light source. The light source may be structured, having bright spots positioned in the form of a two dimensional array, or a matrix. The reflected light from the cornea may then appear as a matrix. If the contact lens and cornea are indented by the magnetic force, a distorted image 5100 of the matrix may be observed. The spot positions may come from a cornea apex change from the reference image 5000. After the pulse 6000 ends, the spots in the image may return to their original positions. By analyzing the differences in spot positions, an indentation profile may be determined, and the IOP may be calculated. In general, the displacements of the spots due to applied magnetic field depend on multiple parameters of the cornea such as thickness, radius, as well as imaging configuration, i.e. position of illuminating spots and camera location and focal properties. A ray tracing simulation along with a mechanical finite element analysis of the corneal indentation may be used to estimate the displacements of the spots in the observed image.

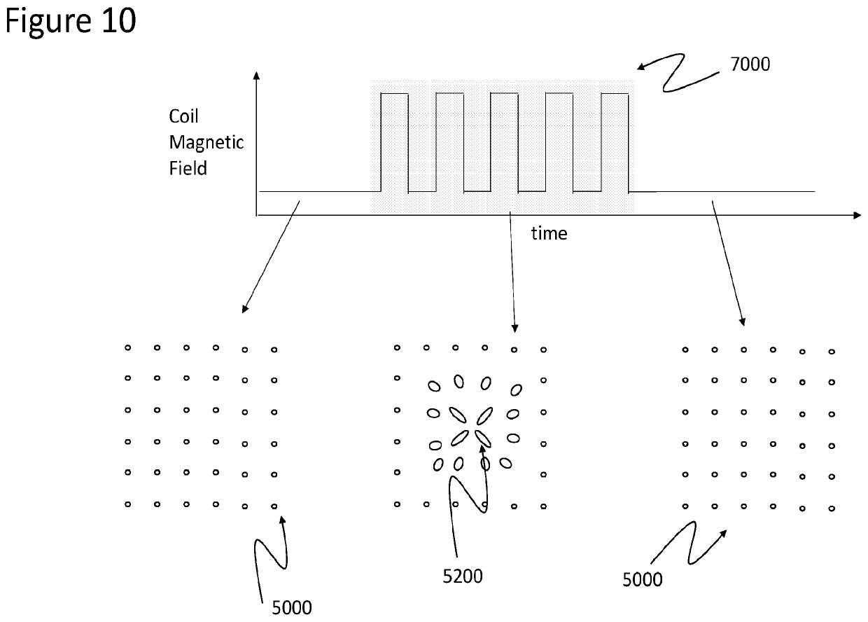

Now referring to FIG. 10, an example measurement of the magnetic force indentation with a periodic magnetic excitation waveform is shown. In an embodiment, a periodic magnetic field pulse train 7000 may be applied to the contact lens through the excitation coil. A camera may record one or more images 5000 of reflections of one or more light sources from the cornea that may appear as a matrix. Upon indentation by the periodic magnetic force at a given frequency, a distorted image 5200 may be observed. The spot positions and their shapes may come from cornea apex changes from the reference image 5000. In some embodiments, the rapidly changing cornea topography may cause a change in the spot shapes in the image 5200 due to a slow response of the camera 6. In some embodiments, the slow response of the camera may be intended. After the pulse 7000 ends, the spots in the image may return to their original positions. By analyzing the differences in spot positions and their shapes, indentation profile at the applied frequency may be inferred. In general, the displacements of the spots due to applied magnetic field may depend on multiple parameters of the cornea such as thickness, radius, as well as the imaging configuration, i.e. position of illuminating spots and camera location and focal properties. A ray tracing simulation along with a mechanical finite element analysis of the corneal indentation may be used to estimate the displacements of the spots in the observed image. Moreover, for a dynamical measurement, the time dependent amplitude of the motion of the cornea may be calculated by a finite element model of the eye. By applying a sequence of frequencies and repeating the measurement, a frequency response plot may be generated for the cornea. Using the frequency plot along with a predetermined look-up table, the IOP may be calculated. Generation of the look-up table may use experimental and computational study that links the cornea anatomical parameters to IOP values. In some embodiments, a frequency dependent measurement may allow the amplitude information to become a relative measurement instead of an absolute measurement and the IOP value may be determined based on the measurement of a resonance frequency instead of a more detailed analysis in the case of absolute deflection measurements.

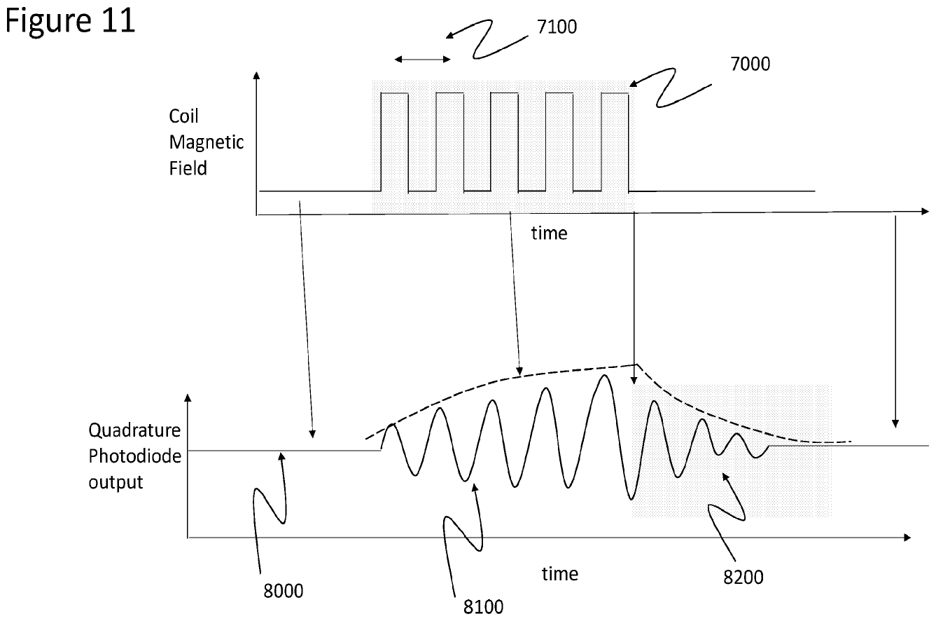

In an embodiment, a graph showing the coil magnetic field vs time is compared to a quadrature photodiode output vs time graph, as seen in FIG. 11. According to an embodiment a periodic magnetic field pulse train 7000 with a known period 7100 may be applied to the contact lens through the excitation coil. The position sensitive photodetector may measure the reflected laser beam deflection, producing a single voltage signal from the detector and may not require a full image capture. Upon indentation by the periodic magnetic force at a given frequency, an exponentially rising signal 8100 may be observed when the pulse train 7000 starts to excite the contact lens. As the pulse train 7000 stops, the mechanical motion may rapidly decay, resulting in an exponentially decaying signal 8200. By analyzing the output signal 8000, 8100, 8200 during different phases of the excitation pulse waveform, the mechanical response at the applied frequency may be inferred. By applying a sequence of frequencies and repeating the measurement, a frequency response plot may be generated for the cornea. Using the frequency plot along with a predetermined look-up table, the IOP may be calculated.

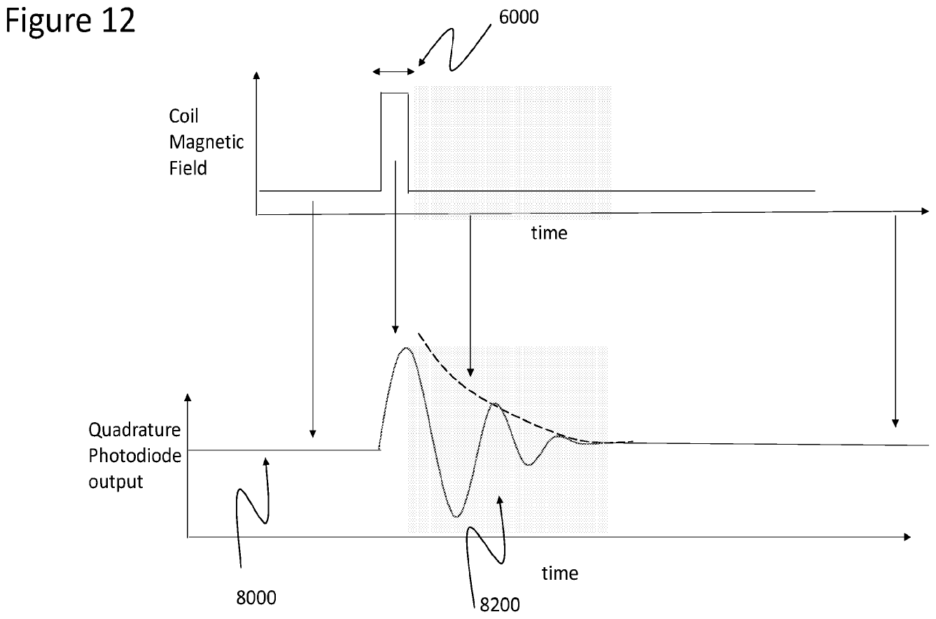

In another embodiment, the measurement of the magnetic force indentation using a single, wideband magnetic excitation waveform may be seen in FIG. 12. The wideband magnetic excitation waveform may be measured by using a laser beam and position sensitive photodetector. A single, short magnetic field pulse 6000 with duration that may be shorter than the typical period of cornea resonance frequency may be applied to the contact lens through the excitation coil. The position sensitive photodetector may measure the reflected laser beam deflection. Upon indentation by the short pulse magnetic force, a wide frequency range may be excited simultaneously. An exponentially rising and decaying signal 8200 may be observed when the pulse train 6000 excites the magnet of the contact lens. As the pulse 6000 stops, the mechanical motion may decay resulting in an exponentially decaying ringdown signal 8200. By analyzing the output signal 8000, 8200 during different phases of the excitation pulse waveform using time series acquisition and Fourier analysis, the mechanical response at all frequencies within the excitation band may be determined. By applying a sequence of pulses and repeating the measurement, signal-to-noise-ratio may be improved and a frequency response plot may be generated for the cornea. Using the frequency plot along with a predetermined look-up table, the IOP may be calculated. Generation of the look-up table may involve study that may link the cornea anatomical parameters to IOP values.

In an embodiment, the motion may be detected through magnetic induction in a readout coil. In an aspect, a single coil may be used for both excitation and pickup. Alternatively, multiple coils for excitation and pickup may be used. According to an embodiment, the motion of the magnet may induce a current at the pickup coil 777, and an electronic amplifier 888 may produce the signal that may be proportional to the displacement of the magnet 2 in the contact lens 1. In some embodiments, this readout method may eliminate the need for optical components. The time dependent signal produced by the inductive pickup method may be proportional to the instantaneous velocity of the magnet during its oscillation.

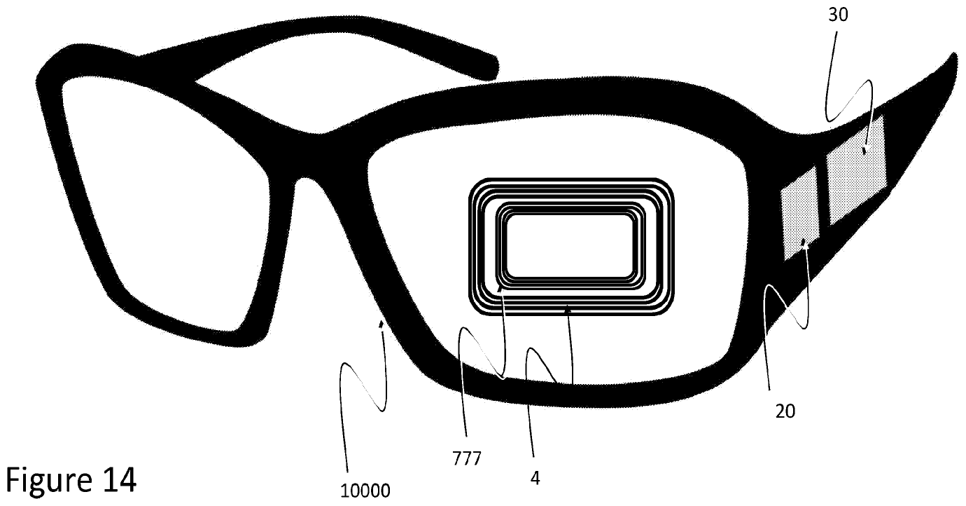

According to yet another embodiment, the electronic readout of the motion of the cornea may be integrated into a pair of goggles, as shown in FIG. 14. In an embodiment, the pickup coil 777 illustrated in FIG. 14 and the excitation coil 4 may be placed on the front of the goggle 10000 in a way that may generally allow vision. The drive electronics and electronic amplifier 888 and the microcontroller 20 that processes the signals may be integrated and placed on the goggle. The system may be powered by external power source or by a battery pack 30, placed on the goggle 10000. In some embodiments, the microcontroller 20 may be a chip, a processor or a computational device programmed with the software to read and analyze data from the magnetic field coils, the image sensors and any other data inputs that may be used to calculate in whole or in part, the IOP of the eye being measured. The microcontroller may be any electronic device suitable for such a purpose, and may include a wired or wireless connection for another computational device, such as a smart phone, tablet computer, laptop, desktop or cloud computer.

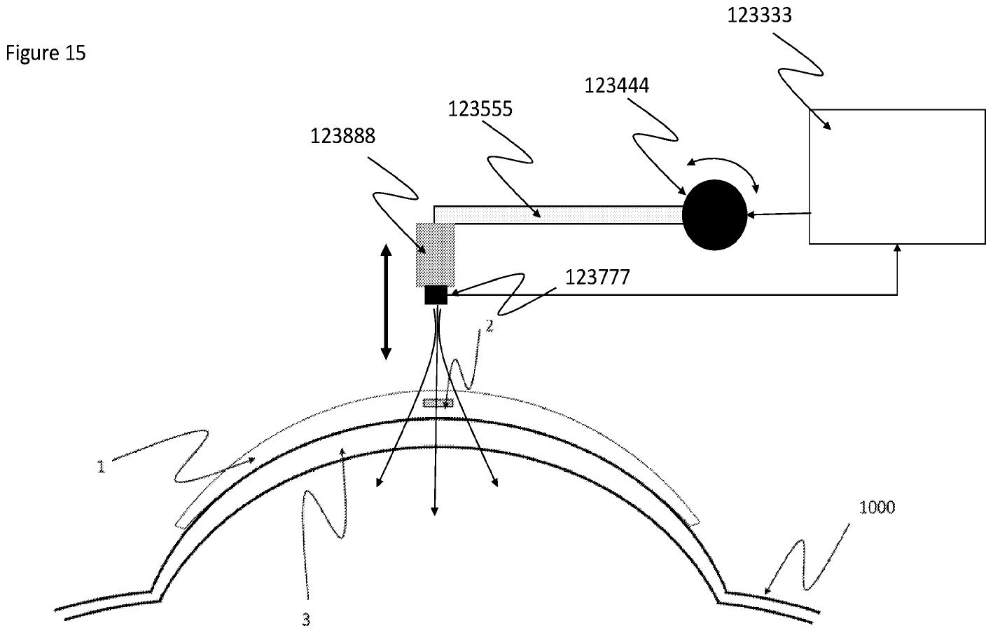

In an embodiment, there may be a system for measuring the electronic readout of the motion of the cornea as shown in FIG. 15. In an embodiment, a magnetic field gradient from a magnet 123888 may cause the motion of the cornea. The magnet may be a permanent magnet or an electromagnet. The magnet 123888 may be poled such that the field emanates from the end and applies a nonlinear force to one or more magnet 2 in or on the contact lens 1. The magnetic field may cause a motion in the contact lens that may correlate with the IOP. A Hall sensor 123777 may be mounted on the magnet 123888. The Hall sensor 123777 output may remain constant, if the magnet 123888 and Hall sensor 123777 move together. In an embodiment. the magnet 2 on the contact lens 1 may be near the Hall sensor 123777, the magnetic field may be perturbed and a signal change may be registered by the controller 123333. The magnet 123888 and Hall sensor 123777 may be mounted on a movable arm 123555 which may be vibrated or otherwise moved by a motion source 123444 such as a galvonometer motion element, and the motion may be controlled by the controller 123333. By recording the field in the Hall sensor 123777 along with the applied motion to the movable arm 123555, the displacement of the cornea may be measured indirectly. Such a measurement may be analyzed and converted into an IOP reading.

In an embodiment, there may be a system for determining an IOP reading. The system comprises a base, an arm, a motor, a magnet, a hall sensor and a controller. In an embodiment, the arm may have a proximal end and a distal end. The proximal end may be in mechanical engagement to the base. The motor may be attached to the arm, the motor may be able to impart motion to the arm. A magnet may be mechanically engaged to the distal end of the arm. The magnet may be capable of producing a controlled magnetic field. The magnet may have a proximal end mechanically engaged to the arm, and a distal end. There may be a hall sensor fixedly attached to the distal end of the magnet. The magnet may generate a directed magnetic field, and the hall sensor may read the magnetic field produced by the magnet. Variations in the magnetic field caused by a contact lens with a magnet may also be read. The determination of the IOP, and control of any electronics, may be performed by a controller.

The advantages of the present disclosure include, without limitation, a robust process for measuring the cornea response through remotely excited microindentation measurement, through the use of a contact lens that houses a magnet and readout optics and/or electronics.

Embodiments of the subject matter and the operations described in this specification may be implemented in digital electronic circuitry, or in computer software, firmware, or hardware, including the structures disclosed in this specification and their structural equivalents, or in combinations of one or more of them. Embodiments of the subject matter described in this specification may be implemented as one or more computer programs, i.e., one or more modules of computer program instructions, encoded on one or more computer storage medium for execution by, or to control the operation of, data processing apparatus, such as a processing circuit. A controller or processing circuit such as CPU may comprise any digital and/or analog circuit components configured to perform the functions described herein, such as a microprocessor, microcontroller, application-specific integrated circuit, programmable logic, etc. Alternatively or in addition, the program instructions may be encoded on an artificially generated propagated signal, e.g., a machine-generated electrical, optical, or electromagnetic signal, that is generated to encode information for transmission to suitable receiver apparatus for execution by a data processing apparatus.

A computer storage medium may be, or be included in, a computer-readable storage device, a computer-readable storage substrate, a random or serial access memory array or device, or a combination of one or more of them. Moreover, while a computer storage medium is not a propagated signal, a computer storage medium may be a source or destination of computer program instructions encoded in an artificially generated propagated signal. The computer storage medium may also be, or be included in, one or more separate components or media (e.g., multiple CDs, disks, or other storage devices). Accordingly, the computer storage medium is both tangible and non-transitory.

The operations described in this specification may be implemented as operations performed by a data processing apparatus on data stored on one or more computer-readable storage devices or received from other sources. The term “data processing apparatus” or “computing device” encompasses all kinds of apparatus, devices, and machines for processing data, including by way of example a programmable processor, a computer, a system on a chip, or multiple ones, or combinations, of the foregoing The apparatus may include special purpose logic circuitry, e.g., an FPGA (field programmable gate array) or an ASIC (application specific integrated circuit). The apparatus may also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment may realize various different computing model infrastructures, such as web services, distributed computing and grid computing infrastructures.

A computer program (also known as a program, software, software application, script, or code) may be written in any form of programming language, including compiled or interpreted languages, declarative or procedural languages, and it may be deployed in any form, including as a standalone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments of the subject matter described in this specification may be implemented on a computer having a display device, e.g., a CRT (cathode ray tube) or LCD (liquid crystal display) monitor, OLED (organic light emitting diode) monitor or other form of display for displaying information to the user and a keyboard and/or a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input. In addition, a computer may interact with a user by sending documents to and receiving documents from a device that is used by the user; for example, by sending web pages to a web browser on a user's client device in response to requests received from the web browser.

While this specification contains many specific embodiment details, these should not be construed as limitations on the scope of any embodiments or of what may be claimed, but rather as descriptions of features specific to particular embodiments. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

Having described certain embodiments of the methods and systems, it will now become apparent to one of skill in the art that other embodiments incorporating the concepts may be used. It should be understood that the systems described above may provide multiple ones of any or each of those components and these components may be provided on either a standalone machine or, in some embodiments, on multiple machines in a distributed system. The systems and methods described above may be implemented as a method, apparatus or article of manufacture using programming and/or engineering techniques to produce software, firmware, hardware, or any combination thereof. In addition, the systems and methods described above may be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMs, RAMs, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture may be accessible from a file server providing access to the computer-readable programs via a network transmission line, wireless transmission media, signals propagating through space, radio waves, infrared signals, etc. The article of manufacture may be a flash memory card or a magnetic tape. The article of manufacture includes hardware logic as well as software or programmable code embedded in a computer readable medium that is executed by a processor. In general, the computer-readable programs may be implemented in any programming language, such as LISP, PERL, C, C++, C#, PROLOG, or in any byte code language such as JAVA. The software programs may be stored on or in one or more articles of manufacture as object code.

What is claimed is:

1. A contact lens device to measure an intraocular pressure of an eye, the device comprising:
a body, wherein the body is formed of an elastomeric material;
at least one magnet embedded in or placed on the body.

2. An apparatus for reading a contact lens with an embedded magnet, the apparatus comprising:
an excitation coil;
a driver in electrical communication with the excitation coil, the driver controlling the parameters of the excitation coil;
a light source, the light source positioned to illuminate the contact lens, wherein the illumination of the contact lens produces a reflection of light, or a refraction of light;
an optical sensor, wherein the optical sensor detects the reflected light or the refracted light, from the contact lens; and
a controller, wherein the controller synchronizes excitation of the coil and sensor data collection, the controller further comprising a receiver to receive information from a first external source.

3. The apparatus of claim 2, wherein the light source is a laser.

4. The apparatus of claim 2, wherein the optical sensor is a camera.

5. The apparatus of claim 2, further comprising an amplifier and digitizer.

6. The apparatus of claim 2, wherein the excitation coil is a magnetic excitation coil.

7. The apparatus of claim 2, wherein the controller further processes one or more of; synthesizes magnetic excitation, optical sensor data collection, a received program instruction from the external source and a received data from the external source.

8. A method of determining a IOP reading using an apparatus for reading a contact lens with an embedded magnet, the apparatus having a controller, the method comprising:
exciting an excitation coil with an electric current pulse;
recording images over a pre-established period of time;
determining a cornea deformation based on one or more cornea topography images; and
calculating the IOP reading using the cornea deformation.

9. The method of claim 8, wherein the excitation of the coil is done with a periodic current pulse train with at least one frequency.

10. The method of claim 8, wherein the cornea topography images further comprises a distortion of an image generated by a light source at one or more frequencies.

11. The method of claim 8, wherein determining the cornea deformation further comprises generating a frequency response curve based on a plurality of cornea deformation images taken at a plurality of frequencies.

12. The method of claim 8, wherein the calculating of the IOP reading further comprises using a computational algorithm to revert a set of corneal frequency response data.

13. The method of claim 8, wherein determining the cornea deformation further comprises measuring the amplitude of signals at an output of a position sensitive photodetector at each frequency.

14. The method of claim 8, wherein determining the cornea deformation further comprises measuring the amplitude of signals at an output coil at each frequency.

15. The method of claim 8, further comprising generating a frequency response curve for a cornea.

16. The method of claim 8, further comprising using a Fourier transform to convert a time domain response to a frequency domain response.

17. The method of claim 16, further comprising calculating a deformation of corneal topography from a signal amplitude at each frequency of the Fourier transform.

18. A system for determining an IOP reading, the system comprising:
a base;
an arm having a proximal end and a distal end, the proximal end being in mechanical engagement to the base;
a motor attached to the arm, the motor able to impart motion to the arm;
a magnet mechanically engaged to the distal end of the arm; the magnet capable of producing a controlled magnetic field, the magnet having a proximal end mechanically engaged to the arm, and a distal end;
a hall sensor fixedly attached to the distal end of the magnet;
wherein the magnet generates a directed magnetic field, and the hall sensor reads the magnetic field produced by the magnet, and at least one contact lens with a magnet, wherein the contact lens with the magnet is in the directed magnetic field.
